(19) United States
(12) Patent Application Publication
(10) Pub. No.: US 2006/0141495 A1
(43) Pub. Date: Jun. 29, 2006

(54) Polymorphic markers and methods of genotyping corn

(76) Inventor: Kunsheng Wu, Ballwin, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/218,305

(22) Filed: Sep. 1, 2005

Related U.S. Application Data
(60) Provisional application No. 60/606,880, filed on Sep. 1, 2004.

(51) Int. Cl. C12Q 1/68; A01H 5/00

(52) U.S. Cl. 435/6; 800/320.1

(57) ABSTRACT

Polymorphic corn DNA loci useful for genotyping between at least two varieties of corn. Sequences of the loci are useful for designing primers and probe oligonucleotides for detecting polymorphisms in corn DNA. Polymorphisms are useful for genotyping applications in corn. The polymorphic markers are useful to establish marker/trait associations, e.g. in linkage disequilibrium mapping and association studies, positional cloning and transgenic applications, marker-aided breeding and marker-assisted selection, and identity by descent studies. The polymorphic markers are also useful in mapping libraries of DNA clones, e.g. for corn QTLs and genes linked to polymorphisms.

### INCORPORATION OF SEQUENCE LISTING

Two copies of the sequence listing (Copy 1 and Copy 2) and a computer readable form (CRF) of the sequence listing, all on CD-ROMs, each containing the file named CornSNP2005.ST25.txt which is 65,830, 912 bytes (measured in MS-DOS), all of which were created on Sep. 01, 2005 are herein incorporated by reference.

### INCORPORATION OF TABLES

Two copies of table, i.e. Table 1 named as CornSNP2005_Table1.txt, on CD-ROMs which is 10,141,696 bytes (measured in MS-Windows), all of which were created on Aug. 18, 2004 are herein incorporated by reference.

### FIELD OF THE INVENTION

Disclosed herein are corn polymorphisms, nucleic acid molecules related to such polymorphisms and methods of using such polymorphisms and molecules, e.g. in genotyping.

### BACKGROUND

Polymorphisms are useful as genetic markers for genotyping applications in the agriculture field, e.g. in plant genetic studies and commercial breeding. See for instance U.S. Pat. Nos. 5,385,835; 5,437,697; 5,385,835; 5,492,547; 5,746,023; 5,962,764; 5,981,832 and 6,100,030, and U.S. applications Ser. No. 09/861,478 (filed May 18, 2001), Ser. No.09/969.373 (filed (Oct. 2, 2001), and Ser. No. 10/389,566 (filed Mar. 14, 2003), the disclosures of all of which are incorporated herein by reference. The highly conserved nature of DNA combined with the rare occurrences of stable polymorphisms provides genetic markers, which are both predictable and discerning of different genotypes. Among the classes of existing genetic markers are a variety of polymorphisms indicating genetic variation including restriction-fragment-length polymorphisms (RFLPs), amplified fragment-length polymorphisms (AFLPs), simple sequence repeats (SSRs), single nucleotide polymorphisms (SNPs) and insertion/deletion polymorphisms (Indels). Because the number of genetic markers for a plant species is limited, the discovery of additional genetic markers will facilitate genotyping applications including marker-trait association studies, gene mapping, gene discovery, marker-assisted selection and marker-assisted breeding. Evolving technologies make certain genetic markers more amenable for rapid, large scale use. For instance, technologies for SNP detection indicate that SNPs may be preferred genetic markers.

### SUMMARY OF THE INVENTION

This invention provides a large number of genetic markers from corn genomic DNA. These genetic markers comprise corn DNA loci, which are useful for genotyping applications involving at least two varieties of corn. A polymorphic corn locus of this invention comprises at least 20 consecutive nucleotides which include or are adjacent to a polymorphism which is identified herein, e.g. in Table 1.

One aspect of this invention is a method of analyzing DNA of a corn plant comprising the steps of obtaining a DNA sequence from a corn line for use as a query; accessing corn DNA sequences having SNP markers including DNA sequences from the Collection of Corn Marker Sequences identified in Table 1, e.g. where the set of polymorphic corn DNA sequences comprises any one of SEQ ID NO:1 through SEQ ID NO: 25043; determining the identity of said query to accessed corn DNA sequences over a window of at least 20 nucleotides; identifying accessed corn DNA sequences having a minimal identity of 90 percent to said query and identifying a SNP marker in said accessed corn DNA sequences; and using the identified SNP marker to genotype the corn line. In one aspect the method of the invention is practiced by accessing corn DNA sequences assembled and stored on a computer readable medium. In genotyping a sequence of DNA extracted from a corn plant is analyzed by comparing the extracted DNA sequence with sequences in a selected set of polymorphic DNA sequences, e.g. to identify polymorphisms in the DNA extracted from a corn plant. In one aspect of the method the selected set comprises all of the DNA sequences of SEQ ID NO: 1 through SEQ ID NO: 25043. In other aspects of the method the selected set can comprise significantly fewer of the polymorphic corn DNA sequences, e.g. a set of limited to a single chromosome or QTL or a set that is relatively evenly distributed over the genome, or a set which is informative for a trait.

Another aspect of this invention provides a method for determining the genotype of a corn plant by analyzing DNA of a corn plant, e.g. by determining the presence of a polymorphic allelic sequence in the DNA of a corn plant, its transcribed mRNA or its translated amino acids and comparing the determined sequence to the sequence of a selected set of polymorphic corn DNA sequences, their transcribed mRNA or translated amino acids. Such comparing allows the identification of allelic character of polymorphisms in the genome of a corn plant. Still another aspect of this invention provides a method for analyzing DNA of a corn plant by assaying DNA from tissue of a corn plant to identify the allelic state of a nucleic acid polymorphism in a polymorphic corn DNA locus identified herein in Table 1. Such assaying can comprise amplifying segments of corn DNA using a pair of oligonucleotide primers designed to hybridize to the 5′ end of each of opposite strands of a segment of corn DNA including a polymorphism which is identified in Table 1. The assaying can further comprise hybridizing an oligonucleotide detector, e.g. having a sequence which hybridizes to the sequence of the DNA at or adjacent to the polymorphism. In such assaying the oligonucleotide primers and oligonucleotide detector can be designed to hybridize to segments of one of the selected set of DNA sequences. A useful assay includes Taqman® assays for SNP detection.

Another aspect of this invention provides a method of analyzing DNA of a corn plant further comprising identifying one or more phenotypic traits for at least two corn lines and determining associations between said traits and polymorphisms.

Still another aspect of this invention is directed to the use of a selected set of polymorphic corn DNA sequences in corn breeding, e.g. by selecting a corn line on the basis of its genotype at a polymorphic locus has a sequence within the selected set of polymorphic corn DNA sequences.

Yet another aspect of this invention provides a method of associating a phenotypic trait to a genotype in a population of corn plants wherein said associating comprises
(a) measuring or characterizing a set of one or more distinct phenotypic traits characterizing the corn plants,
(b) selecting tissue from at least two corn plants having polymorphic DNA and assaying DNA from the tissue to identify the allelic state of a set of distinct polymorphisms, e.g. as identified herein in Table 1, and
(c) identifying associations between the set of allelic states and the set of phenotypic traits,
   where the set of polymorphisms are in loci having sequence in a subset of polymorphic corn DNA sequences. In one aspect of associating the set of polymorphisms comprises at least three, more preferably at least five or more, polymorphisms linked to mapped polymorphisms. A further aspect of the invention contemplates mapping a locus that directly affects a trait of interest by utilizing trait-marker associations discovered using SNP markers disclosed herein, e.g. where the SNP markers are linked to loci permitting disequilibrium mapping of the loci.

Still another aspect of this invention is directed to identifying genes affecting a trait of interest by identifying genes that are genetically or physically linked to a polymorphism wherein said polymorphism is associated with the trait, e.g. using markers of this invention. Such marker/trait association can be useful in marker assisted breeding. More particularly, an aspect of this invention provides a method of corn breeding comprising the steps of
(a) associating an allele of a SNP marker listed in Table 1 with a trait;
(b) genotyping corn lines using said SNP maker,
(c) selecting at least two of said genotyped corn lines which have said allele of a SNP marker;
(d) breeding said selected corn lines to produce progeny.

A further aspect of this invention provides corn plants, including plant parts such as oil, progeny seeds, protein, etc, from corn plant produced by such marker assisted breeding methods.

The methods of this invention characterized by marker identification can be carried out using oligonucleotide primers and oligonucleotides detectors. Thus, another aspect of the invention is directed to such oligonucleotides, e.g. sets of oligonucleotides functional with a marker. More particularly, this invention provides a pair of isolated nucleic acid molecules comprising oligonucleotide primers for amplifying corn DNA to identify the presence of a polymorphism in the DNA, e.g. oligonucleotides comprising at least 12 consecutive nucleotides which are at least 90% identical to ends of a segment of DNA of the same number of nucleotides in opposite strands of a polymorphic corn DNA locus having a sequence which is at least 90% identical to a sequence in a subset of polymorphic corn DNA sequences disclosed herein (or a complement thereof). More preferably such a pair of oligonucleotides comprise at least 15 consecutive nucleotides, or more, e.g. at least 20 consecutive nucleotides. More particularly, when hybridization to a SNP is contemplated for marker assay for identifying a polymorphism in corn DNA, a set will comprise four oligonucleotides, e.g. a pair of isolated nucleic acid molecules for amplifying DNA which can hybridize to DNA which flanks a polymorphism and a pair of detector nucleic acid molecules which are useful for detecting each nucleotide in a single nucleotide polymorphism in a segment of the amplified DNA. In preferred aspects of the invention such detector nucleic acid molecules comprise at least 12 nucleotide bases and a detectable label, or at least 15 nucleotide bases, and the sequence of the detector nucleic acid molecules is identical except for the nucleotide polymorphism (e.g. SNP or Indel) and is at least 95 percent identical to a sequence of the same number of consecutive nucleotides in either strand of the segment of polymorphic corn DNA locus containing the polymorphism.

### DETAILED DESCRIPTION OF PREFERRED EMBODIMENTS

A. Definitions:

As used herein certain terms are defined as follows.

An “allele” means an alternative sequence at a particular locus; the length of an allele can be as small as 1 nucleotide base, but is typically characterized by a longer sequence of common nucleotides bordering the alternative nucleotide. Allelic sequence can be amino acid sequence or nucleic acid sequence. A “locus” is a short sequence that is usually unique and usually found at one particular location in the genome by a point of reference, e.g. a short DNA sequence that is a gene, or part of a gene or intergenic region. A locus of this invention can be a unique PCR product at a particular location in the genome. The loci of this invention comprise one or more polymorphisms i.e. alternative alleles present in some individuals. “Genotype” means the specification of an allelic composition at one or more loci within an individual organism. In the case of diploid organisms, there are two alleles at each locus; a diploid genotype is said to be homozygous when the alleles are the same, and heterozygous when the alleles are different.

“Consensus sequence” means DNA sequence constructed as the consensus at each nucleotide position of a cluster of aligned sequences. Such clusters are used to identify SNP and Indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

“Phenotype” means the detectable characteristics of a cell or organism which are a manifestation of gene expression.

“Marker” means a polymorphic sequence. A “polymorphism” is a variation among individuals in sequence, particularly in DNA sequence. Useful polymorphisms include single base substitutions (single nucleotide polymorphisms SNPs), or insertions or deletions in DNA sequence (Indels) and simple sequence repeats of DNA sequence (SSRs). As used herein “Collection of Corn Marker Sequences” means the set of corn DNA sequences consisting of SEQ ID NO:1 through SEQ ID NO: 25043.

As used herein “Collection of Corn SNP Markers” means the set of corn SNP markers identified in Table 2

“Marker Assay” means an method for detecting a polymorphism at a particular locus using a particular method, e.g. phenotype (such as seed color, flower color, or other visually detectable trait), restriction fragment length polymorphism (RFLP), single base extension, electrophoresis, sequence alignment, allelic specific oligonucleotide hybridization (ASO), RAPID, etc. Preferred marker assays include single base extension as disclosed in U.S. Pat. No. 6,013,431 and allelic discrimination where endonuclease activity releases a reporter dye from a hybridization probe as disclosed in U.S. Pat. No. 5,538,848 the disclosures of both of which are incorporated herein by reference.

“Linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

“Linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium.

“Quantitative Trait Locus (QTL)” means a locus that controls to some degree numerically representable traits that are usually continuously distributed.

Nucleic acid molecules or fragments thereof of the present invention are capable of hybridizing to other nucleic acid molecules under certain circumstances. As used herein, two nucleic acid molecules are said to be capable of hybridizing to one another if the two molecules are capable of forming an anti-parallel, double-stranded nucleic acid structure. A nucleic acid molecule is said to be the “complement” of another nucleic acid molecule if they exhibit “complete complementarity” i.e. each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules which hybridize to other nucleic acid molecules, e.g. at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, New York, 1989 (Now onwards referred as Sambrook et al.) and by Haymes et al., Nucleic Acid Hybridization, A Practical Approach, IRL Press, Washington, DC (1985), each of which is incorporated herein by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, incorporated herein by reference. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid molecule of the present invention will specifically hybridize to one strand of a segment of corn DNA having a nucleic acid sequence as set forth in SEQ ID NO: 1 through SEQ ID NO: 8783 under moderately stringent conditions, for example at about 2.0×SSC and about 65° C., more preferably under high stringency conditions such as 0.2×SSC and about 65° C.

As used herein “sequence identity” refers to the extent to which two optimally aligned polynucleotide or peptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100.

B. Nucleic Acid Molecules—Loci, Primers and Probes

The corn loci of this invention comprise DNA sequence, which comprises at least 20 consecutive nucleotides and includes or is adjacent to one or more polymorphisms identified in Table 1. Such corn loci have a nucleic acid sequence having at least 90% sequence identity, more preferably at least 95% or even more preferably for some alleles at least 98% and in many cases at least 99% sequence identity, to the sequence of the same number of nucleotides in either strand of a segment of corn DNA which includes or is adjacent to the polymorphism. The nucleotide sequence of one strand of such a segment of corn DNA may be found in a sequence in the group consisting of SEQ ID NO: 1 through SEQ ID NO: 25043. It is understood by the very nature of polymorphisms that for at least some alleles there will be no identity at the polymorphic site itself. Thus, sequence identity can be determined for sequence that is exclusive of the polymorphism sequence. The polymorphisms in each locus are identified more particularly in Table 1.

For many genotyping applications it is useful to employ as markers polymorphisms from more than one locus. Thus, one aspect of the invention provides a collection of different loci. The number of loci in such a collection can vary but will be a finite number, e.g. as few as 2 or 5 or 10 or 25 loci or more, for instance up to 40 or 75 or 100 or more loci, e.g. selected because they comprise a set which is limited to a single chromosome or QTL or is relatively evenly distributed over the genome, or is informative for one or more traits.

Another aspect of the invention provides nucleic acid molecules which are capable of hybridizing to the polymorphic corn loci of this invention. In certain embodiments of the invention, e.g. which provide PCR primers, such molecules comprises at least 15 nucleotide bases. Molecules useful as primers can hybridize under high stringency conditions to a one of the strands of a segment of DNA in a polymorphic locus of this invention. Primers for amplifying DNA are provided in pairs, i.e. a forward primer and a reverse primer. One primer will be complementary to one strand of DNA in the locus and the other primer will be complementary to the other strand of DNA in the locus, i.e. the sequence of a primer is preferably at least 90%, more preferably at least 95%, identical to a sequence of the same number of nucleotides in one of the strands. It is understood that such primers can hybridize to sequence in the locus which is distant from the polymorphism, e.g. at least 5, 10, 20, 50 or up to about 100 nucleotide bases away from the polymorphism. Design of a primer of this invention will depend on factors well known in the art, e.g. avoidance of repetitive sequence.

Another aspect of the nucleic acid molecules of this invention are hybridization probes for polymorphism assays. In one aspect of the invention such probes are oligonucleotides comprising at least 12 nucleotide bases and a detectable label. The purpose of such a molecule is to hybridize, e.g. under high stringency conditions, to one strand of DNA in a segment of nucleotide bases which includes or is adjacent to the polymorphism of interest in an amplified part of a polymorphic locus. Such oligonucleotides are preferably at least 90%, more preferably at least 95%, identical to the sequence of a segment of the same number of nucleotides in one strand of corn DNA in a polymorphic locus. The detectable label can be a radioactive element or a dye. In preferred aspects of the invention, the hybridization probe further comprises a fluorescent label and a quencher, e.g. for use hybridization probe assays of the type known as Taqman® assays, available from Applied Biosystems, Foster City, Calif.

For assays where the molecule is designed to hybridize adjacent to a polymorphism which is detected by single base extension, e.g. of a labeled dideoxynucleotide, such molecules can comprise at least 15, more preferably at least 16 or 17, nucleotide bases in a sequence which is at least 90 percent, preferably at least 95%, identical to a sequence of the same number of consecutive nucleotides in either strand of a segment of polymorphic corn DNA. Oligonucleotides for single base extension assays are available from Orchid Biosciences, Inc.

Such primer and probe molecules are generally provided in groups of two primers and one or more probes for use in genotyping assays. Moreover, it is often desirable to conduct a plurality of genotyping assays for a plurality of polymorphisms. Thus, this invention also provides collections of nucleic acid molecules, e.g. in sets which characterize a plurality of polymorphisms.

C. Identifying Polymorphisms

Polymorphisms in a genome can be determined by comparing cDNA sequence from different lines. While the detection of polymorphisms by comparing cDNA sequence is relatively convenient, evaluation of cDNA sequence allows no information about the position of introns in the corresponding genomic DNA. Moreover, polymorphisms in non-coding sequence cannot be identified from cDNA. This can be a disadvantage, e.g. when using cDNA-derived polymorphisms as markers for genotyping of genomic DNA. More efficient genotyping assays can be designed if the scope of polymorphisms includes those present in non-coding unique sequence.

Genomic DNA sequence is more useful than cDNA for identifying and detecting polymorphisms. Polymorphisms in a genome can be determined by comparing genomic DNA sequence from different lines. However, the genomic DNA of higher eukaryotes typically contain a large fraction of repetitive sequence and transposons. Genomic DNA can be more efficiently sequenced if the coding/unique fraction is enriched by subtracting or eliminating the repetitive sequence.

There are a number of strategies that can be employed to enrich for coding/unique sequence. Examples of these include the use of enzymes which are sensitive to cytosine methylation, the use of the McrBC endonuclease to cleave repetitive sequence, and the printing of microarrays of genomic libraries which are then hybridized with repetitive sequence probes.

C.1. methylated cytosine sensitive enzymes: The DNA of higher eukaryotes tends to be very heavily methylated, however it is not uniformly methylated. In fact, repetitive sequence is much more highly methylated than coding sequence. Coding/unique sequence can therefore be enriched by exploiting this difference in methylation pattern. See U.S. Pat. No. 6,017,704 for methods of mapping and assessment of DNA methylation patterns in CG islands. Some restriction endonucleases are sensitive to the presence of methylated cytosine residues in their recognition site. Such methylation sensitive restriction endonucleases may not cleave at their recognition site if the cytosine residue in either an overlapping 5′-CG-3′ or an overlapping 5′-CNG-3′ is methylated. Methylation sensitive restriction endonucleases include the 4 base cutters: Aci I, Hha I, HinP1 I, HpaII and Msp I, the 6 base cutters: Apa I, Age I, Bsr F I, BssH II, Eag I, Eae I, MspM II, Nar I, Pst I, Pvu I, Sac II, Sma I, Stu I and Xho I and the 8 base cutter: Not I. For example, DNA cleavage at the site CTGCAG by Pst I is inhibited when the C residues are methylated. In order to enrich for coding/unique sequence corn libraries can be constructed from genomic DNA digested with Pst I (or other methylation sensitive enzymes), and size fractionated by agarose gel electrophoresis. Regions of the genome which are heavily methylated (i.e., regions with a high fraction of repetitive sequences) have a higher number of Pst I sites that are methylated. Therefore, most of the Pst 1 sites in repetitive DNA will not be cleaved during Pst 1 digestion, and the repetitive sequence will tend to consist mostly of high molecular weight, uncleaved DNA. In contrast, regions of the genome that are not heavily methylated (i.e. regions containing a large fraction of coding/unique sequence) should contain a large fraction of unmethylated Pst I sites which will be cleaved during digestion, producing relatively smaller fragments. When digested DNA is electrophoresed through agarose, relatively larger fragments from heavily methylated, non-coding DNA regions are separated from relatively smaller fragments derived from coding/unique sequence. Coding region-enriched DNA fragments (commonly between 500-3000 bp) can be excised from the gel, purified and ligated into a Pst I digested vector, e.g. pUC18. The ligation products are transformed by electroporation into a plurality of suitable bacterial hosts, e.g. DH10B, to produce a library of clones enriched for coding/unique sequence. Individual clones can be sequenced to provide the sequence of the inserted coding region DNA.

In order to reduce the sequence complexity of any particular library, the DNA in the range 500 to 10,000 bp can be further size-fractionated by incrementally excising fragments from the gel,. Useful ranges of size-fractionated fragments include 500-600 bp, 600-700 bp, 700-800 bp, 800-900 bp, 900-1100 bp, 1100-1500 bp, 1500-2000 bp, 2000-2500 bp and 2500-3000 bp. A series of size-fractionated reduced representation libraries are constructed by ligating purified DNA from each size fraction separately to the vector. A small sample of clones from each library (for example about 400 clones) is sequenced to determine the fraction of repetitive sequence present in each particular library. Comparison of reduced representation libraries prepared from a variety of different corn lines indicates that many fractions contain less than 10% repetitive sequence and some fractions contain more than 20% repetitive sequence. Preferred reduced representation libraries contain less than 20% repetitive sequence, more preferably less than 15% repetitive sequence and even more preferably less than 10% repetitive sequence. By determining the fraction of repetitive sequence throughout the whole series of size fractionated reduced representation libraries, the libraries with the smallest fraction of repetitive sequence can be selected for deep sequencing (usually 10,000-20,000 clones). Since the purpose of obtaining sequence is for polymorphism detection, the equivalent libraries representing the same size fraction for both corn strains are sequenced, or alternatively a library consisting of a mixture of DNA from different corn strains is sequenced. Another advantage of using reduced representation libraries for polymorphism detection is that it increases the probability of recovering the equivalent sequences from both corn lines. Polymorphisms can only be detected if the equivalent sequence is available from both lines.

C.2. McrBC endonuclease

An alternative method for enriching coding region DNA sequence enrichment uses McrBC endonuclease restriction. As a defense against invading foreign DNA from phage/viruses, E. coli contain endonucleases, e.g. McrBC endonuclease, which cleave methylated cytosine-containing DNA. This feature can be exploited to enrich DNA with regions of the genome which are not heavily methylated, e.g. the presumed coding region DNA. Reduced representation libraries can be constructed using genomic DNA fragments which are cleaved by physical shearing or digestion with any restriction enzyme. DNA fragments are transformed into an E.coli host that contains an McrBC endonuclease, e.g. E. coli strain JM107 or DH5a. When the bacterial host is transformed with a DNA fragment which contains methylated DNA region, the McrBC endonuclease will cleave the inserted DNA and the plasmid will not be propagated. When the bacterial host is transformed with a DNA fragment that is not methylated, the plasmid will be propagated, and a colony will grow on the agar plate allowing the clone to be sequenced. A small sample of clones from libraries generated in this manner are sampled, and the fraction of repetitive sequenced determined. McrBC endonuclease can also be used with methylated cytosine sensitive endonuclease to further reduce the fraction of repetitive sequence in libraries that are not suitable for sequencing, e.g. sequences that contain more than 15% repetitive sequence.

C.3. Microarraying Reduced Representation Libraries

Another method to enrich for coding/unique sequence is to construct reduced representation libraries (using methylation sensitive or non-methylation sensitive enzymes), print microarrays of the library on nylon membrane, and hybridize with probes made from repetitive elements known to be present in the library. Clones containing repetitive sequence elements are identified, and the library is re-arrayed by picking only the negative clones. This process is performed by randomly picking clones from a reduced representation library into 384-well plates and culturing them. Micro-arrays can be prepared by printing clone DNA from the collection of 384-well plates in determined patterns on supports, such as glass supports or nylon membranes. The fabrication of microarrays comprising thousands of distinct clones, e.g. up to about 25,000 clones or more, are well known in the art. See for instance, U.S. Pat. No. 5,807,522 for methods for fabricating microarrays of spotted polynucleotides at high density. A small sample of clones from the reduced representation library, e.g. about 400 clones, can be sequenced to identify repetitive sequence elements. Clones containing the repetitive sequences are retrieved, and the clones used to make radioactive probes which are hybridized on the nylon arrays. Radioactive isotope label elements include ³²p, ³³p, 35s, ¹²⁵I, and the like with ³³P being especially preferred. The arrays are analyzed for hybridization by detecting radiation, e.g. using a Fuji Phosphoimager™ imaging screen. After an appropriate exposure time the array image is read as a digital file representing the hybridization intensity from each array element which is proportional to amount of labeled repeat sequence. This radiation image identifies all the clones on the array which correspond to repetitive sequence clones, and also identifies the 384-well plate and well location of each repetitive sequence clone. With this information, all the non-repetitive sequence clones can be picked from the original plates and relocated onto a new set of plates which do not contain repetitive sequence clones. This method can be used to lower the fraction of repetitive sequence in reduced representation libraries from approximately 25% to about 1-2%.

D. Detecting Polymorphisms

Polymorphisms in DNA sequences can be detected by a variety of effective methods well known in the art including those disclosed in U.S. Pat. Nos. 5,468,613 and 5,217,863; 5,210,015; 5,876,930; 6,030,787 6,004,744; 6,013,431; 5,595,890; 5,762,876; 5,945,283; 5,468,613; 6,090,558; 5,800,944 and 5,616,464, all of which are incorporated herein by reference in their entireties. For instance, polymorphisms in DNA sequences can be detected by hybridization to allele-specific oligonucleotide (ASO) probes as disclosed in U.S. Pat. Nos. 5,468,613 and 5,217,863. The nucleotide sequence of an ASO probe is designed to form either a perfectly matched hybrid or to contain a mismatched base pair at the site of the variable nucleotide residues. The distinction between a matched and a mismatched hybrid is based on differences in the thermal stability of the hybrids in the conditions used during hybridization or washing, differences in the stability of the hybrids analyzed by denaturing gradient electrophoresis or chemical cleavage at the site of the mismatch.

U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Length variation in DNA nucleotide sequence repeats such as microsatellites, simple sequence repeats (SSRs) and short tandem repeats (STRs) can be detected by mass spectroscopy methods as disclosed in U.S. Pat. No. 6,090,558 The advantages of using mass spectrometry include a dramatic increase in both the speed of analysis (a few seconds per sample) and the accuracy of direct mass measurements.

Target nucleic acid sequence can also be detected by probe ligation methods as disclosed in U.S. Pat. No. 5,800,944 where sequence of interest is amplified and hybridized to probes followed by ligation to detect a labeled part of the probe.

Target nucleic acid sequence can also be detected by probe linking methods as disclosed in U.S. Pat. No. 5,616,464 employing at least one pair of probes having sequences homologous to adjacent portions of the target nucleic acid sequence and having side chains which non-covalently bind to form a stem upon base pairing of said probes to said target nucleic acid sequence. At least one of the side chains has a photoactivatable group which can form a covalent cross-link with the other side chain member of the stem.

D.1. Primer Base Extension Assay

A preferred method for detecting SNPs and Indels is a labeled base extension method as disclosed in U.S. Pat. Nos. 6,004,744; 6,013,431; 5,595,890; 5,762,876; and 5,945,283. These methods are based on primer extension and incorporation of detectable nucleoside triphosphates. The primer is designed to anneal to the sequence immediately adjacent to the variable nucleotide which can be can be detected after incorporation of as few as one labeled nucleoside triphosphate. The method uses three synthetic oligonucleotides. Two of the oligonucleotides serve as PCR primers and are complementary to sequence of the locus of corn genomic DNA which flanks a region containing the polymorphism to be assayed. Using corn genomic DNA as a template the primer oligonucleotides are used in PCR to produce sufficient copies of the region of the locus containing the polymorphisms so that allelic discrimination can be conducted. Following amplification of the region of the corn genome containing the polymorphism, the PCR product is mixed with the third oligonucleotide (called an extension primer), which is designed to hybridize to the amplified DNA immediately adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

To design primers for corn polymorphism detection by single base extension the sequence of the locus is first masked to prevent design of any of the three primers to sites that match known corn repetitive elements (e.g., transposons) or are of very low sequence complexity (di- or tri-nucleotide repeat sequences). Design of primers to such repetitive elements will result in assays of low specificity, through amplification of multiple loci or annealing of the extension primer to multiple sites.

PCR primers are preferably designed (a) to have an optimal annealing temperature for PCR in the range of 55 to 60° C., (b) to have lengths in the range of 18 to 25 bases, and (c) to produce a product in the size range 75 to 200 base pairs with the polymorphism to be assayed located at least 25 bases from the 3′ end of each primer. The extension primers must be chosen to contain minimal self- or inter-primer complementarity, or the efficiency and/or specificity of the PCR reaction will be reduced.

The extension primer is designed to anneal immediately adjacent to the polymorphism, such that the 3′ end of the annealed extension primer immediately abuts the polymorphic site. The extension primer can lie either to the 5′ or 3′ side of the polymorphism; however, if it is designed to lie on the 3′ side, then the sequence of the extension primer must match the reverse complement of the sequence adjacent to the polymorphism. The extension primer must contain no self-complementarity that will enable self-annealing, or the incorporation of the labeled ddNTPs may result from self-priming of the extension primer, obscuring the results of polymorphism-directed incorporation. If the nature of the sequence adjacent to the polymorphic site makes it impossible to design an extension primer that is fully non-self-complementary, the extent of self-annealing may be limited by replacing one or two bases of the extension primer with abasic sites, as long as the abasic sites are not introduced into the three 3′ most positions.

The labeled ddNTPs chosen for inclusion in the reaction are determined by the nature of the polymorphism, and whether the extension primer lies those that match the first base of the polymorphism. For example, in the case of an AG polymorphism, the ddNTPs would be ddATP-label(1) and ddGTP-label(2) for one strand as template or ddTTP-label(1) and ddCTP-label(2) for the other stand. Labels can be chosen from among a wide variety of chemical moieties, including affinity or immunological labels, fluorescent dyes and mass tags. In the most common embodiment of the process, affinity and immunological labels are used, followed by appropriate detection reagents. In the present example, ddATP-FITC and ddGTP-biotin might be employed, followed by incubation with anti-FITC-antibody conjugated to the enzyme horseradish peroxidase (HRP-anti-FITC), and streptavidin conjugated to the enzyme alkaline phosphatase (AP-streptavidin).

D.2. Labeled Probe Degradation Assay

In another preferred method for detecting polymorphisms SNPs and Indels can be detected by methods disclosed in U.S. Pat. Nos. 5,210,015; 5,876,930 and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter fluorescence, e.g. by Forster-type energy transfer. During PCR forward and reverse primers hybridize to a specific sequence of the target DNA flanking a polymorphism. The hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter. A useful assay is available from Applied Biosystems as the Taqman® assay which employs four synthetic oligonucleotides in a single reaction that concurrently amplifies the corn genomic DNA, discriminates between the alleles present, and directly provides a signal for discrimination and detection. Two of the four oligonucleotides serve as PCR primers and generate a PCR product encompassing the polymorphism to be detected. Two others are allele-specific fluorescence-resonance-energy-transfer (FRET) probes. FRET probes incorporate a fluorophore and a quencher molecule in close proximity so that the fluorescence of the fluorophore is quenched. The signal from a FRET probes is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), (VIC) and 6-carboxyfluorescein phosphoramidite (FAM). A useful quencher is 6-carboxy-N,N,N′,N′-tetramethylrhodamine (TAMRA). Additionally, the 3′ end of each FRET probe is chemically blocked so that it can not act as a PCR primer. During the assay, corn genomic DNA is added to a buffer containing the two PCR primers and two FRET probes. Also present is a third fluorophore used as a passive reference, e.g., rhodamine X (ROX) to aid in later normalization of the relevant fluorescence values (correcting for volumetric errors in reaction assembly). Amplification of the genomic DNA is initiated. During each cycle of the PCR, the FRET probes anneal in an allele-specific manner to the template DNA molecules. Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred.

To design primers and probes for the assay the locus sequence is first masked to prevent design of any of the three primers to sites that match known corn repetitive elements (e.g., transposons) or are of very low sequence complexity (di- or tri-nucleotide repeat sequences). Design of primers to such repetitive elements will result in assays of low specificity, through amplification of multiple loci or annealing of the FRET probes to multiple sites.

PCR primers are designed (a) to have a length in the size range of 18 to 35 bases and matching sequences in the polymorphic locus,(b) to have a calculated melting temperature in the range of 57 to 65° C., e.g. corresponding to an optimal PCR annealing temperature of 52 to 60° C., (c) to produce a product which includes the polymorphic site and has a length in the size range of 75 to 250 base pairs. The PCR primers are preferably located on the locus so that the polymorphic site is at least one base away from the 3′ end of each PCR primer. The PCR primers must not be contain regions that are extensively self- or inter-complementary.

FRET probes are designed to span the sequence of the polymorphic site, preferably with the polymorphism located in the 3′ most ⅔ of the oligonucleotide. In the preferred embodiment, the FRET probes will have incorporated at their 3′ end a chemical moiety which, when the probe is annealed to the template DNA, binds to the minor groove of the DNA, thus enhancing the stability of the probe-template complex. The probes should have a length in the range of 12 to 20 bases, and with the 3′ MGB, have a calculated melting temperature of 5 to 7° C. above that of the PCR primers. Probe design is disclosed in US Pat. Nos. 5,538,848; 6,084,102 and 6,127,121.

E. Construction of Genetic Linkage Maps

Genetic linkage maps can be constructed using the JoinMap version 2.0 software which is described by Stam, P. “Construction of integrated genetic linkage maps by means of a new computer package: JoinMap, The Plant Journal, 3: 739-744 (1993); Stam, P. and van Ooijen, J. W. “JoinMap version 2.0: Software for the calculation of genetic linkage maps (1995) CPRO-DLO, Wageningen. JoinMap implements a weighted-least squares approach to multipoint mapping in which information from all pairs of linked loci (adjacent or not) is incorporated. Linkage groups are formed using a LOD threshold of 5.0.

Alternatively genetic linkage maps can be constructed using the MAPMAKER/EXP v3.0 software described by Landers et al (Lander E. S., Green P., Abrahamson J., Barlow A., Daly M. J., Lincoln S. E., and Newburg I., Genomics 1: 174-181, 1987). MAPMAKER/EXP performs full multipoint linkage analysis (simultaneous estimation of all recombination fractions from the primary data) for dominant, recessive, and co-dominant (e.g. RFLP-like) markers. Public SSRs, e.g. approximately 1 every 20 cM, can be used as frameworks prior to SNP placement on the 20 linkage groups of corn (Cregan P. B., Jarvik T., Bush L., Shoemaker R. C., Lark K. G., Kahler A. L., VanToai T. T., Lohnes D. G., Chung J., Specht J. E., Crop Sci. 39:1464-1490, 1999). MAPMAKER/EXP's “group” command can be used at LOD thresholds of 20.0, 10.0, 5.0, and 3.0 for gross linkage group assignment. Next, “order” command ( LOD threshold 2.0) is used to order markers within the linkage groups. The “try” command is used to place all remaining markers onto the linkage groups. Then the “ripple” command is used to verify local order. (group”, “order”, “try”, and “ripple” commands are described in MAPMAKER/EXP). Centimorgan distance is calculated using the Kosambi or Haldane mapping function. (Kosambi D. D., Ann Eugen. 12: 172-175, 1944; Haldane, J. B. S., J. Genet. 8:299-309, 1919.).

The ordered linkage groups, defined by soft wares JointMap v 2.0 or MAPMAKER/EXP, are arranged in Microsoft Excel in accordance to the software's output. SSR and SNP loci, cM distance ( Kosambi mapping function), and genotypic scores are arranged, from top to bottom, to detect possible errors in scores( double-crossovers and misscores). After verifying genotypic scores for accuracy and consistency, the loci can be once again mapped using JointMap v 2.0 or MAPMAKER/EXP to finalize map order, cM distance, and the addition of previously unmapped loci.

Jansen discloses an alternative approach for linkage map construction based on finding a locus order to minimize the total number of recombination events (Jansen J. et al. in Theor Appl Genet. 102: 1113-1122, 2001 ). Under many conditions this approach yields a close approximation to a maximum-likelihood map. A map estimated by this approach agrees quite closely with the map obtained using JoinMap 2.0

F. Use Of Polymorphisms To Establish Marker/Trait Associations

The polymorphisms in the loci of this invention can be used in marker/trait associations which are inferred from statistical analysis of genotypes and phenotypes of the members of a population. These members may be individual organisms, e.g. corn, families of closely related individuals, inbred lines, dihaploids or other groups of closely related individuals. Such corn groups are referred to as “lines”, indicating line of descent. The population may be descended from a single cross between two individuals or two lines (e.g. a mapping population) or it may consist of individuals with many lines of descent. Each individual or line is characterized by a single or average trait phenotype and by the genotypes at one or more marker loci.

Several types of statistical analysis can be used to infer marker/trait association from the phenotype/genotype data, but a basic idea is to detect markers, i.e. polymorphisms, for which alternative genotypes have significantly different average phenotypes. For example, if a given marker locus A has three alternative genotypes (AA, Aa and aa), and if those three classes of individuals have significantly different phenotypes, then one infers that locus A is associated with the trait. The significance of differences in phenotype may be tested by several types of standard statistical tests such as linear regression of marker genotypes on phenotype or analysis of variance (ANOVA). Commercially available, statistical software packages commonly used to do this type of analysis include SAS Enterprise Miner (SAS Institute Inc., Cary, N.C.) and Splus (Insightful Corporation. Cambridge, Mass.). When many markers are tested simultaneously, an adjustment such as Bonferonni correction is made in the level of significance required to declare an association.

Often the goal of an association study is not simply to detect marker/trait associations, but to estimate the location of genes affecting the trait directly (i.e. QTLs) relative to the marker locations. In a simple approach to this goal, one makes a comparison among marker loci of the magnitude of difference among alternative genotypes or the level of significance of that difference. Trait genes are inferred to be located nearest the marker(s) that have the greatest associated genotypic difference. In a more complex analysis, such as interval mapping (Lander and Botstein, Genetics 121:185-199 (1989), each of many positions along the genetic map (say at 1 cM intervals) is tested for the likelihood that a QTL is located at that position. The genotype/phenotype data are used to calculate for each test position a LOD score (log of likelihood ratio). When the LOD score exceeds a critical threshold value, there is significant evidence for the location of a QTL at that position on the genetic map (which will fall between two particular marker loci).

F.1. Linkage Disequilibrium Mapping and Association Studies

Another approach to determining trait gene location is to analyze trait-marker associations in a population within which individuals differ at both trait and marker loci. Certain marker alleles may be associated with certain trait locus alleles in this population due to population genetic process such as the unique origin of mutations, founder events, random drift and population structure. This association is referred to as linkage disequilibrium. In linkage disequilibrium mapping, one compares the trait values of individuals with different genotypes at a marker locus. Typically, a significant trait difference indicates close proximity between marker locus and one or more trait loci. If the marker density is appropriately high and the linkage disequilibrium occurs only between very closely linked sites on a chromosome, the location of trait loci can be very precise.

A specific type of linkage disequilibrium mapping is known as association studies. This approach makes use of markers within candidate genes, which are genes that are thought to be functionally involved in development of the trait because of information such as biochemistry, physiology, transcriptional profiling and reverse genetic experiments in model organisms. In association studies, markers within candidate genes are tested for association with trait variation. If linkage disequilibrium in the study population is restricted to very closely linked sites (i.e. within a gene or between adjacent genes), a positive association provides nearly conclusive evidence that the candidate gene is a trait gene.

F.2. Positional Cloning and Transgenic Applications

Traditional linkage mapping typically localizes a trait gene to an interval between two genetic markers (referred to as flanking markers). When this interval is relatively small (say less than 1 Mb), it becomes feasible to precisely identify the trait gene by a positional cloning procedure. A high marker density is required to narrow down the interval length sufficiently. This procedure requires a library of large insert genomic clones (such as a BAC library), where the inserts are pieces (usually 100-150 kb in length) of genomic DNA from the species of interest. The library is screened by probe hybridization or PCR to identify clones that contain the flanking marker sequences. Then a series of partially overlapping clones that connects the two flanking clones (a “contig”) is built up through physical mapping procedures. These procedures include fingerprinting, STS content mapping and sequence-tagged connector methodologies. Once the physical contig is constructed and sequenced, the sequence is searched for all transcriptional units. The transcriptional unit that corresponds to the trait gene can be determined by comparing sequences between mutant and wild type strains, by additional fine-scale genetic mapping, and/or by functional testing through plant transformation. Trait genes identified in this way become leads for transgenic product development. Similarly, trait genes identified by association studies with candidate genes become leads for transgenic product development.

F.3. Marker-Aided Breeding and Marker-Assisted Selection

When a trait gene has been localized in the vicinity of genetic markers, those markers can be used to select for improved values of the trait without the need for phenotypic analysis at each cycle of selection. In marker aided breeding and marker-assisted selection, associations between trait genes and markers are established initially through genetic mapping analysis (as in A.1 or A.2). In the same process, one determines which marker alleles are linked to favorable trait gene alleles. Subsequently, marker alleles associated with favorable trait gene alleles are selected in the population. This procedure will improve the value of the trait provided that there is sufficiently close linkage between markers and trait genes. The degree of linkage required depends upon the number of generations of selection because, at each generation, there is opportunity for breakdown of the association through recombination.

Prediction of Crosses for New Inbred Line Development

The associations between specific marker alleles and favorable trait gene alleles also can be used to predict what types of progeny may segregate from a given cross. This prediction may allow selection of appropriate parents to generation populations from which new combinations of favorable trait gene alleles are assembled to produce a new inbred line. For example, if line A has marker alleles previously known to be associated with favorable trait alleles at loci 1, 20 and 31, while line B has marker alleles associated with favorable effects at loci 15, 27 and 29, then a new line could be developed by crossing A x B and selecting progeny that have favorable alleles at all 6 trait loci.

F.4. Fingerprinting and Introgression of Transgenes

A fingerprint of an inbred line is the combination of alleles at a set of marker loci. High density fingerprints can be used to establish and trace the identity of germplasm, which has utility in germplasm ownership protection.

Genetic markers are used to accelerate introgression of transgenes into new genetic backgrounds (i.e. into a diverse range of germplasm). Simple introgression involves crossing a transgenic line to an elite inbred line and then backcrossing the hybrid repeatedly to the elite (recurrent) parent, while selecting for maintenance of the transgene. Over multiple backcross generations, the genetic background of the original transgenic line is replaced gradually by the genetic background of the elite inbred through recombination and segregation. This process can be accelerated by selection on marker alleles that derive from the recurrent parent.

G. Use of Polymorphism Assay for Identifying Gene of Interest.

The polymorphisms and loci of this invention are useful for identifying and mapping DNA sequence of QTLs and genes linked to the polymorphisms. For instance, BAC or YAC clone libraries can be queried using polymorphisms linked to a trait to find a clone containing specific QTLs and genes associated with the trait. For instance, QTLs and genes in a plurality, e.g. hundreds or thousands, of large, multi-gene sequences can be identified by hybridization with an oligonucleotide probe which hybridizes to a mapped and/or linked polymorphism. Such hybridization screening can be improved by providing clone sequence in a high density array. The screening method is more preferably enhanced by employing a pooling strategy to significantly reduce the number of hybridizations required to identify a clone containing the polymorphism. When the polymorphisms are mapped, the screening effectively maps the clones.

For instance, in a case where thousands of clones are arranged in a defined array, e.g. in 96 well plates, the plates can be arbitrarily arranged in three-dimensionally, arrayed stacks of wells each comprising a unique DNA clone. The wells in each stack can be represented as discrete elements in a three dimensional array of rows, columns and plates. In one aspect of the invention the number of stacks and plates in a stack are about equal to minimize the number of assays. The stacks of plates allow the construction of pools of cloned DNA.

For a three-dimensionally arrayed stack pools of cloned DNA can be created for (a) all of the elements in each row, (b) all of the elements of each column, and (c) all of the elements of each plate. Hybridization screening of the pools with an oligonucleotide probe which hybridizes to a polymorphism unique to one of the clones will provide a positive indication for one column pool, one row pool and one plate pool, thereby indicating the well element containing the target clone.

In the case of multiple stacks, additional pools of all of the clone DNA in each stack allows indication of the stack having the row-column-plate coordinates of the target clone. For instance, a 4608 clone set can be disposed in 48 96-well plates. The 48 plates can be arranged in 8 sets of 6 plate stacks providing 6×12×8 three-dimensional arrays of elements, i.e. each stack comprises 6 stacks of 8 rows and 12 columns. For the entire clone set there are 36 pools, i.e. 6 stack pools, 8 row pools, 12 column pools and 8 stack pools. Thus, a maximum of 36 hybridization reactions is required to find the clone harboring QTLs or genes associated or linked to each mapped polymorphism.

Once a clone is identified, oligonucleotide primers designed from the locus of the polymorphism can be used for positional cloning of the linked QTL and/or genes.

H. Computer Readable Media, Databases and Methods

The sequences of nucleic acid molecules of this invention can be “provided” in a variety of mediums to facilitate use, e.g. a database or computer readable medium, which can also contain descriptive annotations in a form that allows a skilled artisan to examine or query the sequences and obtain useful information. In one embodiment of the invention computer readable media may be prepared that comprise nucleic acid sequences where at least 10% or more, e.g. at least 25%, or even at least 50% or more of the sequences of the loci and nucleic acid molecules of this invention. For instance, such database or computer readable medium may comprise sets of the loci of this invention or sets of primers and probes useful for assaying the polymorphisms of this invention. In addition such database or computer readable medium may comprise a figure or table of the mapped or unmapped polymorphisms or this invention and genetic maps.

As used herein “database” refers to any representation of retrievable collected data including computer files such as text files, database files, spreadsheet files and image files, printed tabulations and graphical representations and combinations of digital and image data collections. In a preferred aspect of the invention, “database” means a memory system that can store computer searchable information. Currently, preferred database applications include those provided by DB2, Sybase and Oracle.

As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to the result of a process for storing information in a retrievable database or computer readable medium. For instance, a skilled artisan can readily adopt any of the presently known methods for recording information on computer readable medium to generate media comprising the mapped polymorphisms and other nucleotide sequence information of the present invention. A variety of data storage structures are available to a skilled artisan for creating a computer readable medium where the choice of the data storage structure will generally be based on the means chosen to access the stored information. In addition, a variety of data processor programs and formats can be used to store the polymorphisms and nucleotide sequence information of the present invention on computer readable medium.

Computer software is publicly available which allows a skilled artisan to access sequence information provided in a computer readable medium. The examples which follow demonstrate how software which implements a search algorithm such as the BLAST algorithm (Altschul et al., J. Mol. Biol. 215:403-410 (1990), incorporated herein by reference) and the BLAZE algorithm (Brutlag et al., Comp. Chem. 17:203-207 (1993), incorporated herein by reference) on a Sybase system can be used to identify DNA sequence which is homologous to the sequence of loci of this invention with a high level of identity. Sequence of high identity can be compared to find polymorphic markers useful with corn varieties.

The present invention further provides systems, particularly computer-based systems, which contain the sequence information described herein. Such systems are designed to identify commercially important sequence segments of the nucleic acid molecules of this invention. As used herein, “a computer-based system refers to the hardware, software and memory used to analyze the nucleotide sequence information. A skilled artisan can readily appreciate that any one of many currently available computer-based systems are suitable for use in practicing the present invention by a computer-based method.

As indicated above, it is preferable to practice the methods of this invention using computer-based systems comprising a database having stored therein polymorphic markers, genetic maps, and/or the sequence of nucleic acid molecules of the present invention and the necessary hardware and software for supporting and implementing genotyping applications.

### EXAMPLE 1

This example illustrates identification of SNP and Indel polymorphisms by comparing alignments of the sequences of contigs and singletons from at least two separate maize lines. Genomic and cDNA libraries from multiple maize lines were made by isolating genomic DNA or mRNA from different maize lines by Plant DNAzol Reagent or RNAzol” from Life Technologies now Invitrogen (Invitrogen Life Technologies, Carlsbad, Calif.). For genomic libraries, genomic DNA were digested with Pst 1 endonuclease restriction enzyme, size fractionated over 1% agarose gel and ligated in plasmid vector for sequencing by standard molecular biology techniques as described in Sambrook et al. cDNA libraries were made by using “SuperScript™ plasmid system for cDNA synthesis and plasmid cloning” kits from Life Technologies now Invitrogen (Invitrogen Life Technologies, Carlsbad, Calif.) by following manufacturers' instructions. These libraries were sequenced by standard procedures on ABI Prism®377 DNA Sequencer using commercially available reagents (Applied Biosystems, Foster City, Calif.). All sequences are assembles to identify non redundant sequences by Pangea Clustering and Alignment Tools which is available from DoubleTwist Inc., Oakland, Calif. Difference in sequences from multiple clones on assembles contigs is identified as single or multiple nucleotide polymorphism. Sequence from multiple maize lines is assembled to into loci having one or more polymorphisms, i.e. SNPs and/or Indels. Candidate polymorphisms are qualified by the following parameters:
(a) The minimum length of a contig or singleton for a consensus alignment is 200 bases.
(b) The percentage identity of observed bases in a region of 15 bases on each side of a candidate SNP, is at least 75%.
(c) The minimum sequence reads in a given contig is 4.

A plurality of loci having qualified polymorphisms are identified as having consensus sequence as reported as SEQ ID NO: 1 through SEQ ID NO:25043. Qualified SNP and Indel polymorphisms in each locus are identified in Table 1. More particularly, Table 1 identifies the type and location of the polymorphisms as follows:

SEQ ID NO: refers to the sequence number of the polymorphic maize DNA locus of the invention, e.g. a SEQ ID NO.

SEQUENCE NAME refers to an arbitrary name for identifying the polymorphic maize DNA locus.

LENGTH refers to the length of the consensus sequence.

SNP_ID refers to an arbitrary name for identifying each polymorphism.

POSITION refers to the position in the nucleotide sequence of the polymorphic maize DNA locus where the polymorphism occurs.

“A” refers to the total counts of sequence reads in the contig that contain an “A” at the position specified at “POSITION” column. A also refers to nucleoside Adenosine.

“C” refers to the total counts of sequence reads in the contig that contain a “C” at the position specified at “POSITION” column. C also refers to nucleoside Cytosine.

“G” refers to the total counts of sequence reads in the contig that contain a “G” at the position specified at “POSITION” column. G also refers to nucleside Guanosine.

“T” refers to the total counts of sequence reads in the contig that contain a “T” at the position specified at “POSITION” column. T also refers to nucleoside Thymidine.

“-” refers to the total counts of sequence reads in the contig that contain a missing base or nucleoside at the position specified at “POSITION” column.

### EXAMPLE 2

This example illustrates the use of primer base extension for detecting a SNP polymorphism. Reference is made ZmSNP2004_{—}11516_{—}2_c3430, in the polymorphic maize locus of SEQ ID NO: 969. Three polymorphisms in that locus are described more particularly in the following Table 2A which is extracted from Table 1.

TABLE 2A

| SEQ ID | | START | END | | ALLELE 1/ | ALLELE 2/ |
|---|---|---|---|---|---|---|
| NO: | SNP_ID | Position | Position | TYPE | STRAIN 1 | STRAIN 2 |
| 969 | ZmSNP2004_11516_2_c3430 | 3430 | 3430 | SNP | C/MO17 | G/B73 |
| 969 | ZmSNP2004_11516_2_a3535 | 3535 | 3535 | SNP | A/MO17 | G/B73 |
| 969 | ZmSNP2004_11516_2_a3784 | 3784 | 3784 | SNP | A/MO17 | G/B73 |

TABLE 2B

| | | | | |
|---|---|---|---|---|
| Description | Name | Probe | SNP | Sequence |

| | | | | |
|---|---|---|---|---|
| PCR primer | 969-3430 F | | | GGTTTGATCTTCCTGCTT |
| | | | | TGGA |
| PCR primer | 969-3430 R | | | CACCAAACATATTGAATA |
| | | | | CTGGCTTT |
| SNP probe | 969-3430V | VIC | C | ATACGCCTTCGCTCA |
| SNP probe | 969-3430 M | FAM | G | TACGCGTTCGCTCA |

With reference to Table 2B, forward and reverse PCR primers (“969-3430F” and “969-3430R”) and reporter dye-tagged probes (“969-3430V” and “969-3430M”) are designed to hybridize to template DNA sequence in the polymorphic maize DNA locus of SEQ ID NO: 969 around the C/G SNP polymorphism of SNP_ID: ZmSNP2004_{—}11516_{—}2_c3430. Such probes can be designed and provided by Applied Biosystems for their proprietary Taqman® assay (Applied Biosystems, Foster City, Calif.).

A quantity of maize genomic template DNA (e.g. about 2-20 nanograms) is mixed in 5 microliter total volume with four oligonucleotides, i.e. “969-3430F” forward primer, “969-3430R” reverse primer, “969-3430V” SNP hybridization probe having a VIC reporter attached to the 5′ end, and “969-3430M” SNP hybridization probe having a FAM reporter attached to the 5′ end with appropriate amount of PCR reaction buffer containing the passive reference dye ROX. The PCR reaction is conducted for 35 cycles using a 60° C. annealing-extension temperature. Following the reaction, the fluorescence of each fluorophore as well as that of the passive reference is determined in a fluorimeter. The fluorescence value for each fluorophore is normalized to the fluorescence value of the passive reference. The normalized values are plotted against each other for each sample to produce an allelogram. A successful genotyping assay using the primers and hybridization probes of this example provides an allelogram with data points in clearly separable clusters.

To confirm that an assay produces accurate results, each new assay is performed on a number of replicates of samples of known genotypic identity representing each of the three possible genotypes, i.e. two homozygous alleles and a heterozygous sample. To be a valid and useful assay, it must produce clearly separable clusters of data points, such that one of the three genotypes can be assigned for at least 90% of the data points, and the assignment is observed to be correct for at least 98% of the data points. Subsequent to this validation step, the assay is applied to progeny of a cross between two highly inbred individuals to obtain segregation data, which are then used to calculate a genetic map position for the polymorphic locus.

What is claimed is:

1-18. (canceled)

19. A set of four oligonucleotides useful for identifying a polymorphism in corn DNA identified in Table 1 comprising
(a) a pair of isolated nucleic acid molecules according to claim 16 which can hybridize to DNA which flanks a polymorphism identified in Table 1;
(b) a pair of detector nucleic acid molecules which are useful for detecting each nucleotide in a single nucleotide polymorphism in a segment of DNA amplified by said pair of nucleic acid molecule primers of (a), wherein said detector nucleic acid molecules comprise
(1) at least 12 nucleotide bases and a detectable label, or
(2) at least 15 nucleotide bases, and wherein the sequence of said detector nucleic acid molecules is identical except for said nucleotide polymorphism and is at least 95 percent identical to a sequence of the same number of consecutive nucleotides in either strand of said segment of polymorphic corn DNA locus said polymorphism.
